(19) United States
(12) Patent Application Publication
(10) Pub. No.: US 2010/0198278 A1
(43) Pub. Date: Aug. 5, 2010

(54) COMPOSITE ANTIMICROBIAL ACCESSORY INCLUDING A MEMBRANE LAYER AND A POROUS LAYER

(75) Inventors: Kenneth E. Cobian, St. Anthony, MN (US); Genevieve L. Gallagher, Mendota Heights, MN (US); James L. Schuld, Plymouth, MN (US); Michael B. Shelton, Golden Valley, MN (US); Peter M. Seiler, St. Anthony Village, MN (US); Michael S. Hemenway, Mounds View, MN (US)

Correspondence Address: Medtronic, Inc., 710 Medtronic Parkway, Minneapolis, MN, 55432, US

(73) Assignee: Medtronic, Inc., Minneapolis, MN (US)

(21) Appl. No.: 12/698,866

(22) Filed: Feb. 2, 2010

Related U.S. Application Data
(60) Provisional application No. 61/149,214, filed on Feb. 2, 2009.
(60) Provisional application No. 61/152,467, filed on Feb. 13, 2009.
(60) Provisional application No. 61/165,273, filed on Mar. 31, 2009.
(60) Provisional application No. 61/218,328, filed on Jun. 18, 2009.
(60) Provisional application No. 61/256,758, filed on Oct. 30, 2009.

(51) Int. Cl. A61N 1/375; B32B 3/26; B32B 37/00; B32B 38/08

(52) U.S. Cl. 607/2; 428/304.4; 156/280; 156/242

(57) ABSTRACT

An antimicrobial accessory may include a membrane layer defining a major surface comprising a first lateral portion, a second lateral portion, and a third lateral portion. The membrane layer may comprise a first biodegradable polymer. The antimicrobial accessory may further include a first porous layer overlying the first lateral portion and a second porous layer overlying the second lateral portion. The first porous layer comprises a second biodegradable polymer and a first antimicrobial, while the second porous layer comprises a third biodegradable polymer and a second antimicrobial.

This application claims the benefit of U.S. Provisional Application No. 61/149,214, entitled, “ANTIMICROBIAL ACCESSORY FOR AN IMPLANTABLE MEDICAL DEVICE,” filed on Feb. 2, 2009, U.S. Provisional Application No. 61/152,467, entitled, “ANTIMICROBIAL ACCESSORY INCLUDING A POROUS POLYMER LAYER,” filed on Feb. 13, 2009, U.S. Provisional Application No. 61/165,273, entitled, “ANTIMICROBIAL ACCESSORY FOR AN IMPLANTABLE MEDICAL DEVICE,” filed on Mar. 31, 2009, U.S. Provisional Application No. 61/218,328, entitled, “PATTERNED ANTIMICROBIAL ACCESSORY FOR AN IMPLANTABLE MEDICAL DEVICE,” filed Jun. 18, 2009, and U.S. Provisional Application No. 61/256,758, entitled, “COMPOSITE ANTIMICROBIAL ACCESSORY INCLUDING A MEMBRANE LAYER AND A POROUS LAYER,” filed Oct. 30, 2009, the entire contents of which are incorporated herein by reference.

### TECHNICAL FIELD

The disclosure relates to implantable medical devices and, more particularly, to techniques for reducing risk of post-implantation infection.

### BACKGROUND

Implantable medical devices (IMDs) include a variety of devices that provide therapy (such as electrical simulation or drug delivery) to a patient, monitor a physiological parameter of a patient, or both. IMDs typically include a number of functional components encased in a housing. The housing is implanted in a body of the patient. For example, the housing may be implanted in a pocket created in a torso of a patient. The housing may be constructed of a biocompatible material, such as titanium. While the housing is biocompatible, there may still be a risk of infection to the patient as a result of the implantation procedure or the presence of the IMD in the body.

### SUMMARY

In general, the disclosure is directed to an antimicrobial accessory for an implantable medical device (IMD) and techniques for manufacturing the antimicrobial accessory. The antimicrobial accessory may be configured to be attached to or implanted adjacent to the IMD to promote wound healing and/or reduce or substantially eliminate risk of post-implant infection to a patient in which the IMD is implanted. The antimicrobial accessory may include a membrane layer, a first porous layer overlying a first portion of the membrane layer, and a second porous layer overlying a second portion of the membrane layer. Each of the first porous layer and the second porous layer may include an antimicrobial disposed in pores of the porous layer. One example of a polymer in the porous layer is collagen, and one example of an antimicrobial is gentamicin.

In one aspect, the disclosure is directed to an antimicrobial accessory including a membrane layer defining a major surface comprising a first lateral portion and a second lateral portion, where the membrane layer comprises a first biodegradable polymer. According to this aspect of the disclosure, the antimicrobial accessory may further include a porous layer overlying the first lateral portion, and the porous layer may comprise a second biodegradable polymer and an antimicrobial. Additionally, in this aspect of the disclosure, the porous layer does not overlie the second lateral portion of the membrane layer.

In another aspect, the disclosure is directed to a kit including an implantable medical device and an antimicrobial accessory. According to this aspect of the disclosure, the antimicrobial accessory may include a membrane layer defining a first major surface comprising a first lateral portion and a second lateral portion. The antimicrobial accessory may further include a porous layer overlying the first lateral portion. According to this aspect of the disclosure, the membrane layer comprises a first biodegradable polymer and the porous layer comprises a second biodegradable polymer and an antimicrobial.

In an additional aspect, the disclosure is directed to a method including forming a porous layer comprising a first biodegradable polymer and forming a membrane layer comprising a second biodegradable polymer. According to this aspect of the disclosure, a major surface of the membrane layer comprises a first lateral portion and a second lateral portion. The method may further include coupling the porous layer to the first lateral portion, where the porous layer does not overlie the second lateral portion, and depositing an antimicrobial in pores of the porous layer.

In a further aspect, the disclosure is directed to a method including forming a first porous layer comprising a first biodegradable polymer, forming a second porous layer comprising a second biodegradable polymer, and forming a membrane layer comprising a third biodegradable polymer. A major surface of the membrane layer may comprise a first lateral portion, a second lateral portion, and a third lateral portion. According to this aspect of the disclosure, the method further comprises coupling the first porous layer to the first lateral portion, coupling the second porous layer to the second lateral portion, and depositing an antimicrobial in pores of the first porous layer and the second porous layer.

In an additional aspect, the disclosure is directed to a method including forming a porous layer comprising a first biodegradable polymer and an antimicrobial and forming a membrane layer comprising a second biodegradable polymer. According to this aspect of the disclosure, a major surface of the membrane layer comprises a first lateral portion and a second lateral portion. This aspect of the disclosure further includes coupling the porous layer to the first lateral portion, and the porous layer does not overlie the second portion.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example therapy system that may be used to provide cardiac stimulation therapy to a patient, and which includes an antimicrobial accessory.

FIG. 2 is a cross-sectional diagram illustrating an antimicrobial accessory including a membrane layer, a first porous layer overlying a first portion of the membrane layer, and a second porous layer overlying a second portion of the membrane layer.

FIG. 3 is a flow diagram illustrating an example of method by which an antimicrobial accessory is formed.

FIG. 4A is a conceptual diagram illustrating an antimicrobial accessory applied around a portion of an implantable medical device.

FIG. 4B is a conceptual diagram illustrating an antimicrobial accessory applied to a portion of an implantable medical device.

FIG. 5A is a conceptual diagram illustrating an antimicrobial accessory applied to a portion of an implantable medical device.

FIG. 5B is a conceptual diagram illustrating an antimicrobial accessory applied around a portion of an implantable medical device.

FIG. 6 is a conceptual diagram illustrating an example of an antimicrobial accessory including a membrane layer and a porous polymer layer.

FIG. 7 is a flow diagram illustrating an example of a method by which an antimicrobial accessory is formed.

### DETAILED DESCRIPTION

In general, the disclosure is directed to an antimicrobial accessory for an implantable medical device (IMD). The antimicrobial accessory may be configured to be attached to or implanted adjacent to the IMD to promote wound healing and/or to reduce or substantially eliminate risk of infection proximate to an implant site at which the IMD is implanted in a body of a patient. The antimicrobial accessory may include a membrane layer and a porous layer overlying a first lateral portion of the membrane layer. A second lateral portion of the membrane layer may not include an overlying porous layer. In some examples, the membrane layer may include a surface defining a first lateral portion, a second lateral portion and a third lateral portion. In such examples, the antimicrobial may include first and second porous layers overlying the first lateral portion and the second lateral portion, respectively. In any case, the porous layer(s) include an antimicrobial disposed in pores of the porous layer(s). One example of a polymer in the porous layer is collagen, and one example of an antimicrobial is gentamicin.

Such an antimicrobial accessory may provide advantages. For example, the membrane layer of the antimicrobial accessory may wrap around at least a portion of an IMD and may facilitate handling and implantation of the antimicrobial accessory. In some examples, wrapping the membrane layer of the antimicrobial accessory around at least a portion of the IMD may assist in maintaining relative positioning between the IMD and the antimicrobial accessory, i.e., the membrane layer may function as a fixation mechanism. In examples in which the antimicrobial includes a first and a second porous layer, the first and second porous layers may facilitate delivery of the antimicrobial to substantially an entire implantation pocket or implantation site. The antimicrobial accessory according to the present disclosure may also reduce a volume occupied by the accessory compared to an antimicrobial accessory that comprises a sponge layer substantially throughout the accessory. Additionally or alternatively, the antimicrobial accessory may provide desirable handling characteristics due to use of a membrane layer in addition to sponge layers. In some embodiments, the membrane layer may provide desirable mechanical properties to the antimicrobial accessory during handling and implantation of the antimicrobial accessory.

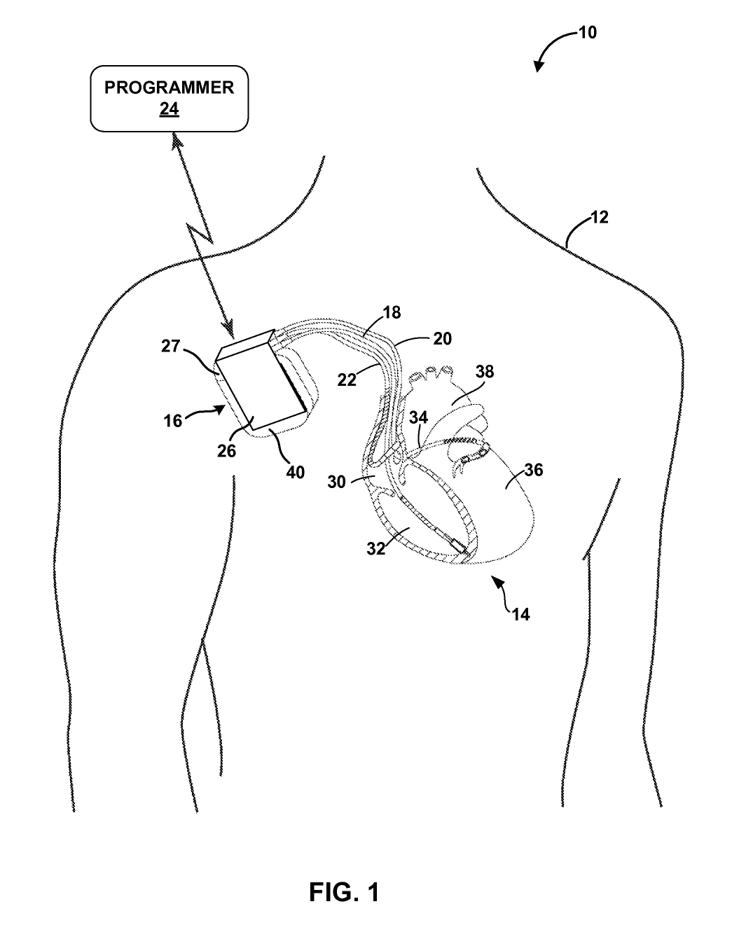

FIG. 1 is a conceptual diagram illustrating an example therapy system 10 that may be used to provide therapy to a patient 12. Patient 12 ordinarily, but not necessarily, will be a human. Therapy system 10 may include an implantable cardiac device (ICD) 16 and a programmer 24. In the example illustrated in FIG. 1, ICD 16 has an antimicrobial accessory 26 attached to a surface of a housing 40 of ICD 16.

While the examples in the disclosure are primarily directed to an antimicrobial accessory 26 attached to an ICD 16, in other examples, antimicrobial accessory 26 may be utilized with other implantable medical devices. For example, antimicrobial accessory 26 may be attached to an implantable drug delivery device, an implantable monitoring device that monitors one or more physiological parameter of patient 12, an implantable neurostimulator (e.g., a spinal cord stimulator, a deep brain stimulator, a pelvic floor stimulator, a peripheral nerve stimulator, or the like), a cardiac or neurological lead, a catheter, an orthopedic device such as a spinal device, or the like. In general, antimicrobial accessory 26 may be attached to or implanted proximate to any medical device configured to be implanted in a body of a patient 12.

In the example depicted in FIG. 1, ICD 16 is connected (or “coupled”) to leads 18, 20, and 22. ICD 16 may be, for example, a device that provides cardiac rhythm management therapy to heart 14, and may include, for example, an implantable pacemaker, cardioverter, and/or defibrillator that provides therapy to heart 14 of patient 12 via electrodes coupled to one or more of leads 18, 20, and 22. In some examples, ICD 16 may deliver pacing pulses, but not cardioversion or defibrillation shocks, while in other examples, ICD 16 may deliver cardioversion or defibrillation shocks, but not pacing pulses. In addition, in further examples, ICD 16 may deliver pacing pulses, cardioversion shocks, and defibrillation shocks.

Leads 18, 20, 22 that are coupled to ICD 16 extend into the heart 14 of patient 12 to sense electrical activity of heart 14 and/or deliver electrical stimulation to heart 14. In the example shown in FIG. 1, right ventricular (RV) lead 18 extends through one or more veins (not shown), the superior vena cava (not shown), and right atrium 30, and into right ventricle 32. Left ventricular (LV) coronary sinus lead 20 extends through one or more veins, the vena cava, right atrium 30, and into the coronary sinus 34 to a region adjacent to the free wall of left ventricle 36 of heart 14. Right atrial (RA) lead 22 extends through one or more veins and the vena cava, and into the right atrium 30 of heart 14. In other examples, ICD 16 may deliver stimulation therapy to heart 14 by delivering stimulation to an extravascular tissue site in addition to or instead of delivering stimulation via electrodes of intravascular leads 18, 20, 22.

ICD 16 may sense electrical signals attendant to the depolarization and repolarization of heart 14 (e.g., cardiac signals) via electrodes (not shown in FIG. 1) coupled to at least one of the leads 18, 20, 22. In some examples, ICD 16 provides pacing pulses to heart 14 based on the cardiac signals sensed within heart 14. The configurations of electrodes used by ICD 16 for sensing and pacing may be unipolar or bipolar. ICD 16 may also provide defibrillation therapy and/or cardioversion therapy via electrodes located on at least one of the leads 18, 20, 22. ICD 16 may detect arrhythmia of heart 14, such as fibrillation of ventricles 32 and 36, and deliver defibrillation therapy to heart 14 in the form of electrical shocks. In some examples, ICD 16 may be programmed to deliver a progression of therapies, e.g., shocks with increasing energy levels, until a fibrillation of heart 14 is stopped. ICD 16 may detect fibrillation by employing one or more fibrillation detection techniques known in the art. For example, ICD 16 may identify cardiac parameters of the cardiac signal, e.g., R-waves, and detect fibrillation based on the identified cardiac parameters.

In some examples, programmer 24 may be a handheld computing device or a computer workstation. Programmer 24 may include a user interface that receives input from a user. The user interface may include, for example, a keypad and a display, which may be, for example, a cathode ray tube (CRT) display, a liquid crystal display (LCD) or light emitting diode (LED) display. The keypad may take the form of an alphanumeric keypad or a reduced set of keys associated with particular functions. Programmer 24 can additionally or alternatively include a peripheral pointing device, such as a mouse, via which a user may interact with the user interface. In some embodiments, a display of programmer 24 may include a touch screen display, and a user may interact with programmer 24 via the display.

A user, such as a physician, technician, or other clinician, may interact with programmer 24 to communicate with ICD 16. For example, the user may interact with programmer 24 to retrieve physiological or diagnostic information from ICD 16. A user may also interact with programmer 24 to program ICD 16, e.g., select values for operational parameters of ICD 16.

Programmer 24 may communicate with ICD 16 via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the ICD 16 implant site in order to improve the quality or security of communication between ICD 16 and programmer 24.

Antimicrobial accessory 26 may be attached to at least two surfaces of housing 40 and/or connector block 27 or may be implanted adjacent to at least two surfaces of ICD 16. Antimicrobial accessory 26 may wrap around at least a portion of ICD 16. In other words, antimicrobial accessory 26 may be disposed simultaneously on or about at least two sides or surfaces of ICD 16. For example, FIG. 1 illustrates an example in which antimicrobial accessory 26 is wrapped around three sides of ICD 16 (a first major side, a top, and a second major side of ICD 16). In some examples, antimicrobial accessory 26 may be wrapped around more than three sides of ICD 16. Additionally and optionally, antimicrobial accessory 26 may be wrapped around a portion of at least one of leads 18, 20, 22.

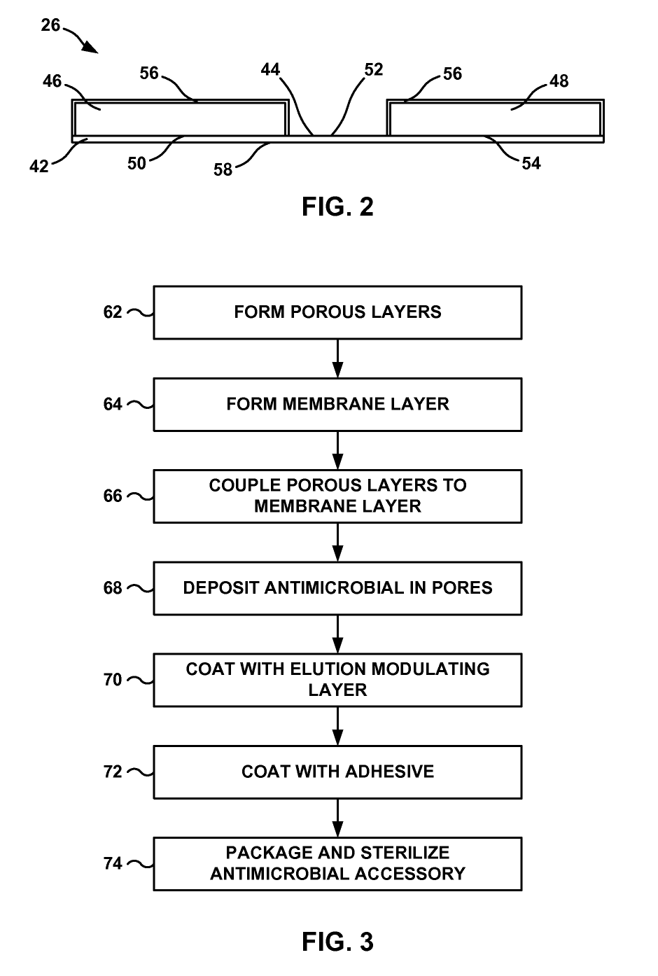

FIG. 2 illustrates further details of one example of antimicrobial accessory 26. Antimicrobial accessory 26 may include a membrane layer 42, a first porous layer 46 formed on a first major surface 44 of the membrane layer 42, and a second porous layer 48 formed on first major surface 44. Membrane layer 42 may be less porous (or more dense) than first porous layer 46 and second porous layer 48. In some examples, membrane layer 42 may be substantially nonporous.

Membrane layer 42 may define a first major surface 44 and a second major surface 58 substantially opposite first major surface 44. First major surface 44 may include a first lateral portion 50, second lateral portion 52, and third lateral portion 54. First and second porous layers 46, 48 may be formed on first lateral portion 50 and third lateral portion 54, respectively, and a second lateral portion 52 of first major surface 44 may not include a porous layer formed thereon. The first and second porous layers 46, 48 of antimicrobial accessory 26 may include an antimicrobial disposed in pores of the porous layers 46, 48. The antimicrobial disposed in pores of first porous layer 46 may be the same as or different than the antimicrobial disposed in pores of second porous layer 48. In some examples, at least one of first porous layer 46 and second porous layer 48 may include at least two antimicrobials.

In some examples, membrane layer 42 may also include an antimicrobial, which may be the same as or different than the antimicrobial disposed in the pores of porous layers 46, 48. Membrane layer 42 may include substantially no antimicrobial, a lower concentration of antimicrobial than at least one of first porous layer 46 and second porous layer 48, or a greater concentration of antimicrobial than at least one of first porous layer 46 and second porous layer 48. By including an antimicrobial in membrane layer 42, greater control of an elution profile of the antimicrobial(s) in membrane layer 42, first porous layer 46, and second porous layer 48 may be achieved compared to including antimicrobial(s) in only first porous layer 46 and second porous layer 48. For example, because membrane layer 42 is less porous than first porous layer 46 and second porous layer 48, the antimicrobial in membrane layer 42 may elute more slowly than antimicrobial in first porous layer 46 and/or second porous layer 48.

The antimicrobial in first porous layer 46, second porous layer 48, and/or membrane layer 42 may include, for example, an antibiotic such as tetracyclines (e.g., minocycline, doxycycline), rifamycins (e.g., rifampin, rifaximin, rifapentine, rifabutin), macrolides (e.g., erythromycin), penicillins (e.g., nafcillin), cephalosporins (e.g., cefazolin), other beta-lactam antibiotics (e.g., imipenem, aztreonam) aminoglycosides (e.g., gentamicin), glycopeptides (e.g., vancomycin, teicoplanin), quinolones (e.g., ciprofloxacin), fusidic acid, trimethoprim, metronidazole, mupirocin, polenes (e.g., amphotericin B), azoles (e.g., fluconazole) and beta-lactam inhibitors (e.g., sulbactam), tigecycline, daptomycin, clindamycin, or another fluoroquinolone, bacitracin, neomycin, an antiseptic, an antimicrobial peptide, a quaternary ammonium, or the like. In some examples, the antimicrobial may be provided in a salt form, e.g., gentamicin crobefate or gentamicin sulfate. In some examples, two or more antimicrobials may be selected to efficaciously prevent or treat any infection present proximate to the implant location of ICD 16, e.g., infection in the pocket in which ICD 16 is implanted. For example, gentamicin may be utilized alone or in combination with at least one other antimicrobial.

Each of membrane layer 42, first porous layer 46, and second porous layer 48 may include a biodegradable or bioabsorbable polymer, such as, for example, at least one of collagen, poly(lactic-co-glycolic acid) (PLGA), poly(lactic acid) (PLA), poly(glycolic acid) (PGA), poly(ethylene oxide) (PEO), poly(ortho ester) (POE), poly(ε-caprolactone) (PCL), poly(dioxanone), polyglyconate, hyaluronic acid, gelatin, fibrin, fibrinogen, cellulose, starch, cellulose acetate, polyvinylpyrrolidone (PVP), a poly(ethylene oxide)/poly(propylene oxide) copolymer (PEO-PPO), poly(ethylene vinyl acetate), poly(hydroxybutyrate-covalerate), polyanhydride, poly(glycolic acid-co-trimethylene carbonate), polyphosphoester, polyphosphoester urethane, a poly(amino acid), a cyanoacrylate, poly(trimethylene carbonate), poly(iminocarbonate), a copoly(ether-ester) such as PEO/PLA, a polyalkylene oxalate, a polyphasphazene, a polyarylate, a tyrosine-based biodegradable or bioabsorbable polymer, poly hydroxyalkanoate (PHA), a sugar ester, or the like. In some examples, at least one of membrane layer 42, first porous layer 46, or second porous layer 48 includes a mixture of at least two polymers. In some cases, at least one of membrane layer 42, first porous layer 46, and second porous layer 48 may include a different polymer than another of membrane layer 42, first porous layer 46, and second porous layer 48. In other examples, membrane layer 42, first porous layer 46, and second porous layer 48 may comprise the same polymer. The biodegradable or bioabsorbable polymer may degrade and be absorbed by the body of patient 12 over time after implantation of antimicrobial accessory 26 in the body of patient 12. This may be advantageous as it may ensure that substantially all of the antimicrobial is released from antimicrobial accessory 26, which may reduce risk of the growth or development of organisms that are resistant to the antimicrobial. Further, absorption of antimicrobial accessory 26 over time may remove a location for bacteria to grow.

In some examples, each of membrane layer 42, first porous layer 46, and second porous layer 48 may comprise collagen, alone or in combination with at least one other polymer. The collagen may comprise collagen from an animal (xenogenous collagen) or from a human (autologous or allogenic collagen). The collagen may comprise at least one collagen type, such as Type-I, -II, -III, -IV, -VII, or -IX. Collagen Type-I may be obtained from animal tissue such as skin, tendons, or the like. In some examples, the collagen may be enzymatically treated prior to use, while in other examples, the collagen may not be enzymatically treated prior to use. Collagen may or may not be cross-linked.

Regardless of the particular polymer from which antimicrobial accessory 26 is formed, antimicrobial accessory 26 may include other components that may influence the properties of the accessory 26. For example, antimicrobial accessory 26 may include an antioxidant mixed in the polymer and/or the antimicrobial, which may reduce or substantially prevent oxidation of the antimicrobial. Exemplary antioxidants include, but are not limited to, monofunctional hindered phenolic antioxidants, such as those available under the trade designations Irganox 1076 and Irganox 1010 from Ciba Corp., Tarrytown, N.Y., butylated hydroxyl toluene (BHT), vitamin E, vitamin A, or vitamin C. In some examples, antimicrobial accessory 26 may include between approximately 0.1 weight percent (wt. %) and approximately 2 wt. % antioxidant.

Antimicrobial accessory 26 may optionally include an elution modulating layer 56 formed on at least one of first porous layer 46 or second porous layer 48. Elution modulating layer 56 may affect the rate at which antimicrobial is released from the porous layer 46 or 48 on which elution modulating layer 56 is formed. In some examples, elution modulating layer 56 may comprise the same polymer as the porous layer 46, 48 on which elution modulating layer 56 is formed, while in other examples, the elution modulating layer 56 includes a different polymer than the porous layer 46, 48 on which elution modulating layer 56 is formed. For example, the elution modulating layer may include a biodegradable polymer such as collagen, a polysaccharide, or the like. Elution modulating layer 56 may be applied to at least one of first porous layer 46 or second porous layer 48 by a variety of coating techniques, including, for example, spray coating, dip coating, extrusion, or the like.

Elution modulating layer 56 may affect the elution rate of the antimicrobial from first and/or second porous layer 46, 48 in one or more ways. In some examples, elution modulating layer 56 may simply increases a distance through which the antimicrobial must diffuse in order to be released into patient 12, thus delaying initial release of the antimicrobial from antimicrobial accessory 26. As another example, the antimicrobial in the first and/or second porous layer 46, 48 may be relatively insoluble (or have a relatively low solubility) in elution modulating layer 56 such that diffusion of the antimicrobial through elution modulating layer 56 is relatively slow. In addition, the polymer from which the elution modulating layer 56 is formed may also affect the elution rate of the antimicrobial. For example, a dissolvable polymer, such as a polysaccharide, may result in a faster elution rate than a polymer such as polyurethane, because the polysaccharide may dissolve after antimicrobial accessory 26 is implanted in a body of a patient, while a polyurethane elution modulating layer 56 will not dissolve.

The thickness and/or porosity of the elution modulating layer also may be controlled to control the rate at which the antimicrobial elutes from the porous polymer layer. For example, a less porous elution modulating layer 56 may reduce the rate at which the antimicrobial elutes from the porous polymer layer 46 or 48 compared to a more porous elution modulating layer 56. As another example, a thicker elution modulating layer 56 may reduce the rate at which the antimicrobial elutes from the porous polymer compared to a thinner elution modulating layer 56 or no elution modulating layer 56. In some examples, the thickness of the elution modulating layer 56 may range from approximately 100 nm to approximately 1 mm. In other examples, the thickness of the elution modulating layer 56 may range from approximately 100 nm to approximately 10 μm.

In some examples, elution modulating layer 56 may include an antimicrobial mixed in the polymer. The antimicrobial may be the same antimicrobial as in first porous layer 46 and/or second porous layer 48. In some examples, elution modulating layer 56 may include the same antimicrobial as the porous layer 46 or 48 which elution modulating layer 56 covers.

In some examples, elution modulating layer 56 may include an amount of antimicrobial to provide an initial release of antimicrobial after implantation of antimicrobial accessory 26 in patient 12. Elution modulating layer 56 may include no antimicrobial, less antimicrobial than the porous layer on which elution modulating layer 56 is formed, a similar amount of antimicrobial as the porous layer on which elution modulating layer 56 is formed, or more antimicrobial than the porous layer on which elution modulating layer 56 is formed. By utilizing or not utilizing elution modulating layer 56, and by including or not including antimicrobial in elution modulating layer 56, the elution profile of antimicrobial from antimicrobial accessory 26 may be controlled.

Although not shown in FIG. 2, in some examples, second major surface 58 of membrane layer 42 or a surface of first porous layer 46 and/or second porous layer 48 may include formed thereon a layer of adhesive, such as a pressure sensitive adhesive (PSA). Suitable PSAs include, for example, silicone, acrylic, or polyisobutylene PSAs. In some examples, the PSA may be a bioresorbable polymer such as cyanoacrylate, a PLGA-based PSA, or the like. The adhesive layer may be applied to second major surface 58 or a surface of first porous layer 46 and/or second porous layer 48 by, for example, spray coating, knife coating, air knife coating, gap coating, gravure coating, slot die coating, metering rod coating, doctor blade, or the like. The adhesive layer may assist in attaching antimicrobial accessory 26 to ICD 16. In other examples, antimicrobial accessory 26 may be self-adhesive (e.g., a polymer from which at least one of membrane layer 42, first porous layer 46, and/or second porous layer 48 is formed may have adhesive properties when wet), antimicrobial accessory 26 may form a friction fit with surfaces of ICD 16, or antimicrobial accessory 26 may be attached to ICD 16 via a suture or other physical connection. In some examples, antimicrobial accessory 26 may not be attached to ICD 16 and may instead be implanted adjacent to ICD 16.

FIG. 3 illustrates an example of a technique according to which antimicrobial accessory 26 may be formed. Initially, first porous layer 46 and/or second porous layer 48 may be formed as porous sponges (62). In some examples, as described above, first porous layer 46 and second porous layer 48 comprise collagen. In such an example, first porous layer 46 and/or second porous layer 48 may be formed by first creating a dispersion or suspension comprising collagen in a solvent, such as water, another non-organic solvent, or an organic solvent. The dispersion or suspension may include between approximately 0.5 wt. % collagen and approximately 5 wt. % collagen and a balance solvent. The dispersion or suspension then may be freeze dried to form the collagen sponge. The porosity of the collagen sponge may be influenced by the concentration of collagen in the suspension or dispersion. For example, a suspension or dispersion including a higher concentration of collagen may result in a less porous (or denser) collagen sponge. Conversely, a suspension or dispersion including a lower concentration of collagen may result in a more porous (or less dense) collagen sponge.

Next, membrane layer 42 is formed (64). Membrane layer 42 may in some cases comprise collagen, as described above. In some examples in which membrane layer 42 comprises collagen, membrane layer 42 may be formed by applying mechanical pressure and heat to a collagen sponge wetted with an amount of solvent, such as water. For example, a collagen sponge may be wetted such that the solvent content is between approximately 2 wt. % and approximately 40 wt. % of the wetted sponge. The sponge may then be exposed to a temperature between approximately 50° C. and approximately 200° C. at a pressure between approximately 0.5 kg/cm² and approximately 1000 kg/cm² for between approximately 0.1 second and approximately 1 hour to reduce porosity of the sponge and form membrane layer 42.

The porosity of membrane layer 42 may be influenced by the temperature, pressure, and time at which the sponge is pressed. For example, pressing the sponge at a higher pressure, at a higher temperature, and/or for a longer time may result in a membrane layer 42 that is less porous than a sponge pressed at a lower pressure, a lower temperature, and/or a shorter time. Additionally or alternatively, the porosity of membrane layer 42 may also be affected by porosity of the initial sponge which is pressed to form membrane layer 42. As described above, the porosity of the sponge may be influenced by a concentration of collagen in the suspension or dispersion from which the sponge is formed.

In other examples in which membrane layer 42 comprises collagen, membrane layer 42 may be formed directly from a suspension or dispersion of collagen in a solvent. Again, the suspension or dispersion may comprise between approximately 0.5 wt. % and approximately 5 wt. % collagen and a balance solvent, such as water, another non-organic solvent, or an organic solvent. The suspension or dispersion may then be air dried to remove substantially all the solvent and form membrane layer 42. The porosity of the membrane layer 42 in such examples may be influenced by concentration of collagen in the suspension or dispersion, drying conditions, or the like.

First porous layer 46 and second porous layer 48 then may be coupled to membrane layer 42 (66). In some examples, first porous layer 46 and second porous layer 48 may be coupled to membrane layer 42 through use of pressure and elevated temperatures. Similar to the process described above for forming membrane layer 42, first porous layer 46, second porous layer 48, and membrane layer 42 may be wetted with a solvent, such as water, to a moisture content of between approximately 2 wt. % and approximately 40 wt. % of the wetted sponge. First porous layer 46 then may be aligned with and disposed on first lateral portion 50 of membrane layer 42 and second porous layer 48 may be aligned with and disposed on third lateral portion 54 of membrane layer 42. Membrane layer 42, first porous layer 46, and second porous layer 48 may then be exposed to heat and pressure, e.g., by use of a heated press. In some examples, first porous layer 46, second porous layer 48, and membrane layer 42 may then be exposed to a temperature between approximately 50° C. and approximately 200° C. at a pressure between approximately 0.5 kg/cm² and approximately 1000 kg/cm² for between approximately 0.1 second and approximately 1 hour to reduce physically mechanically couple first porous layer 46 to membrane layer 42 and second porous layer 48 to membrane layer 42.

Although the above description implies that first porous layer 46 and second porous layer 48 may be coupled to membrane layer 42 in a single step, in some examples, first porous layer 46 may be coupled to membrane layer 42 in a first step and second porous layer 48 may be coupled to membrane layer 42 in a second step. Such a process may facilitate independent control of the parameters (e.g., pressure, temperature, and time) used to couple first porous layer 46 to membrane layer 42 and second porous layer 48 to membrane layer 42, respectively.

In some examples, the process of attaching first porous layer 46 and second porous layer 48 to membrane layer 42 may reduce a porosity of first porous layer 46 and/or second porous layer 48. However, by appropriately selecting the pressure, temperature, and time for which first porous layer 46 and/or second porous layer 48 are pressed to membrane layer 42, the extent of the porosity reduction of first porous layer 46 and/or second porous layer 48 may be controlled. Controlling an amount of porosity of first porous layer 46 and second porous layer 48 may facilitate control of the extent to which the porous layers 46, 48 swell when exposed to bodily fluids, and may also influence an amount of antimicrobial with which the porous layers 46, 48 may be loaded.

Once first porous layer 46 and second porous layer 48 have been coupled to membrane layer 42, antimicrobial may be deposited in pores of at least one of first porous layer 46 and second porous layer 48 (68). For example, the antimicrobial may be dissolved in a solvent or mixture of solvents at an elevated temperature. The solvent may include, for example, water, methanol, ethanol, isopropyl alcohol, ethyl acetate, acetone, tetrahydrofuran, acetonitrile, heptane, methylene chloride, chloroform, or the like. Antimicrobial accessory 26 including first porous layer 46 and/or second porous layer 48 is then exposed to the solution of the antimicrobial in the solvent by, for example, submerging the porous layer 46 or 48 in the antimicrobial solution or spraying the antimicrobial solution on first porous layer 46 and/or second porous layer 48. Once the pores of porous layers 46, 48 are substantially filled with solution, the solution may be cooled, which initiates precipitation of the antimicrobial out of solution and deposition of the antimicrobial into the pores. Antimicrobial accessory 26 then may be dried in a vacuum to remove substantially all of the solvent.

In some examples, the same antimicrobial may be deposited in pores of each of first porous layer 46 and second porous layer 48. In other examples, a first antimicrobial may be deposited in pores of first porous layer 46 and a second antimicrobial may be deposited in second porous layer 48. In examples in which a different antimicrobial is deposited in each of first porous layer 46 and second porous layer 48, two deposition steps are required, and two antimicrobial solutions may be used. For example, first porous layer 46 may be exposed to a first antimicrobial solution in a first step and second porous layer 48 may be exposed to a second antimicrobial solution in a second step.

In some examples, membrane layer 42 may also comprise an antimicrobial. For example, an antimicrobial may be deposited in membrane layer 42 during the same process during which the antimicrobial is deposited in at least one of first porous layer 46 and second porous layer 48. In other examples, membrane layer 42 may not include an antimicrobial, and layer 42 may not be exposed to the antimicrobial solution when the antimicrobial is deposited in first porous layer 46 and/or second porous layer 48.

Although not shown in FIG. 3, in some embodiments, when forming first porous layer 46 and/or second porous layer 48, the antimicrobial may be mixed in the solvent with the polymer. In this way, antimicrobial may be loaded in first porous layer 46 and/or second porous layer 48 in the same step in which the porous layer 46 and/or 48 is formed, instead of requiring a second step.

In some examples, an elution modulation layer 56 optionally may be formed over at least one of first porous layer 46 and second porous layer 48 (70). Elution modulating layer 56 may be applied to at least one surface of at least one of first porous layer 46 and second porous layer 48, and may affect the rate at which antimicrobial is released from the porous layer 46 or 48 on which elution modulating layer 56 is formed. In some examples, elution modulating layer 56 may comprise the same polymer as the porous layer 46, 48 on which elution modulating layer 56 is formed, while in other examples, the elution modulating layer 56 includes a different polymer than the porous layer 46, 48 on which elution modulating layer 56 is formed. For example, the elution modulating layer may include a biodegradable polymer such as one of those listed above. In some examples, elution modulation layer 56 may comprise collagen, a polysaccharide, or the like. Elution modulating layer 56 may be applied to at least one of first porous layer 46 or second porous layer 48 by a variety of coating techniques, including, for example, spray coating, dip coating, extrusion, or the like.

Additionally or alternatively, a layer of adhesive optionally may be formed on a surface of antimicrobial accessory 26 (72). For example, as described above, the layer of adhesive may be formed on second major surface 58 of membrane layer 42 or a surface of first porous layer 46 and/or second porous layer 48. The adhesive may be a pressure sensitive adhesive (PSA), such as, for example, a silicone, an acrylic, a polyisobutylene PSA, a cyanoacrylate, a PLGA-based PSA, or the like. The adhesive may be applied to a second major surface 58 or a surface of first porous layer 46 and/or second porous layer 48 by, for example, spray coating, knife coating, air knife coating, gap coating, gravure coating, slot die coating, metering rod coating, doctor blade, or the like. The adhesive may assist in attaching antimicrobial accessory 26 to ICD 16. When antimicrobial accessory 26 includes a layer of adhesive applied to second major surface 58 or a surface of first porous layer 46 and/or second porous layer 48, accessory 26 may be disposed on a release liner, such as a fluoropolymer release liner, to provide a convenient article for storing, shipping, and providing to the implanting clinician.

Regardless of whether antimicrobial accessory 26 includes a layer of adhesive, antimicrobial accessory 26 may be packaged in a foil package or other substantially air and water impermeable package that is vacuum sealed or backfilled with an inert gas. Antimicrobial accessory 26 may then be sterilized by, for example, electron beam, gamma beam, ethylene oxide, autoclaving, or the like (74).

In some examples, antimicrobial accessory 26 may be bundled together in a kit with an ICD 16, but may be provided physically separately, e.g., may require the implanting clinician to attach antimicrobial accessory 26 to ICD 16 before implantation. This may provide convenience of having an antimicrobial accessory 26 provided with an ICD 16, but may still permit an implanting clinician to elect if he or she wishes to utilize the antimicrobial accessory 26 on a patient-by-patient basis. Additionally, this may give the implanting clinician discretion in the configuration of antimicrobial accessory 26 relative to ICD 16, i.e., in attaching antimicrobial accessory 26 to ICD 16. For example, the implanting clinician may wrap antimicrobial accessory 26 over various sides of ICD 16 in different orientations, may wrap antimicrobial accessory 26 around at least one lead in addition to or as alternative to wrapping antimicrobial accessory over ICD 16, or the like.

In other examples, an ICD 16 may be provided to the implanting clinician with antimicrobial accessory 26 already attached. This may provide the most straightforward implementation, as the implanting physician is not required to decide whether the antimicrobial accessory 26 is desired, and does not need to attach antimicrobial accessory 26 to ICD 16 prior to implanting ICD 16 in patient 12.

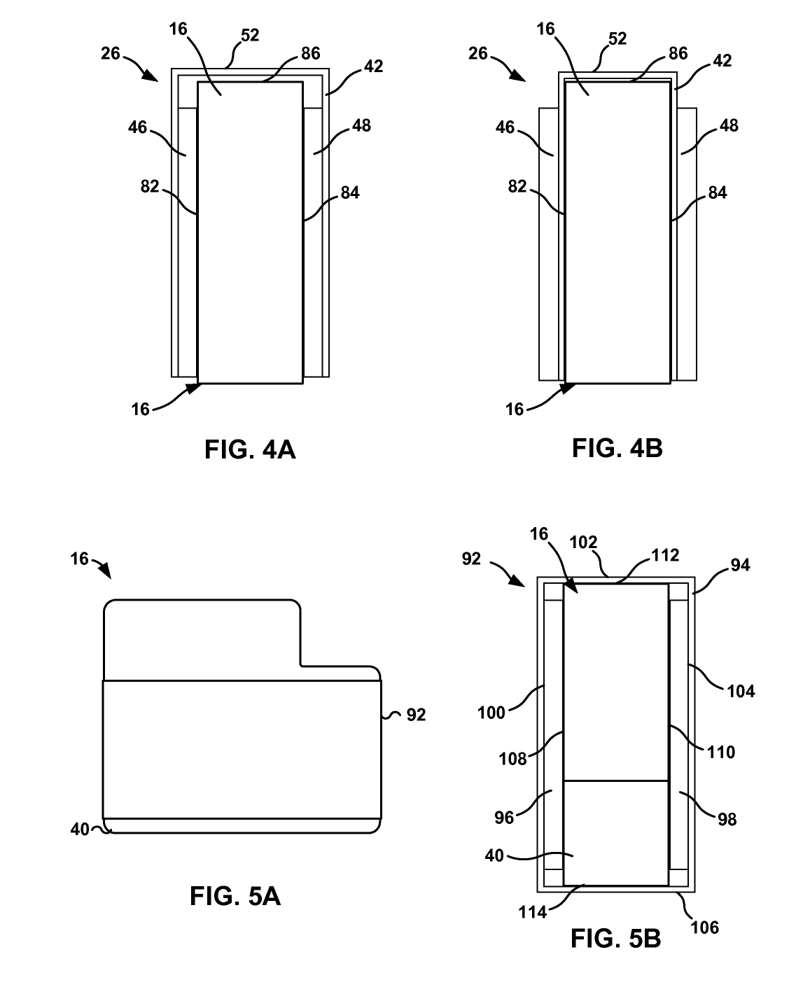

FIGS. 4A and 4B illustrate two examples according to which antimicrobial accessory 26 may be attached to or implanted adjacent to ICD 16. In the example illustrated in FIG. 4A, antimicrobial accessory 26 may be attached to or implanted adjacent to ICD 16 with first porous layer 46 proximate to a first surface 82 of ICD 16 and second porous layer 48 proximate to a second surface 84 of ICD 16. FIG. 4A additionally illustrates an example in which first porous layer 46 is located between membrane layer 42 and first surface 82 and second porous layer 48 located between membrane layer 42 and second surface 84. Second lateral portion 52 of membrane layer 42 may be located proximate a third surface 86 of ICD 16. The configuration of antimicrobial accessory 26 in FIG. 4A may be referred to as “wrapped” about a portion of ICD 16.

In the example illustrated in FIG. 4A, membrane layer 42 may act similar to an elution modulating layer 56 and may affect an elution rate of antimicrobial from first porous layer 46 and/or second porous layer 48 into the surrounding environment of the body of patient 12 (e.g., the implant pocket). Such an arrangement may be beneficial when relatively long-term elution of antimicrobial into the body of patient 12 is desired.

As seen from FIGS. 4A and 4B, antimicrobial accessory 26 may be used to position first porous layer 46 on one side of ICD 16 and second porous layer 48 on a second side of ICD 16. This positioning may be advantageous compared to a configuration in which an antimicrobial accessory is positioned substantially only on one side of ICD 16. For example, antimicrobial accessory 26 may facilitate more uniform spatial delivery of antimicrobial to an implant pocket in which ICD 16 and antimicrobial accessory 26 are implanted. Additionally, the construction of antimicrobial accessory 26 may facilitate handling and implantation of accessory 26 in such an orientation compared to two discrete antimicrobial accessories which are implanted on either side of ICD 16. Membrane layer 42 may be mechanically stronger or tougher than porous layers 46, 48, which may facilitate implantation of antimicrobial accessory 26 and/or may reduce migration of antimicrobial accessory 26 from the initial implantation orientation or position. In some examples, antimicrobial accessory 26 may occupy a relatively smaller volume in the implant pocket than an antimicrobial accessory including a porous layer substantially throughout the antimicrobial accessory, as the portion or portions of antimicrobial accessory 26 that does not include a porous layer 46, 48 (e.g., the second lateral portion 52 of membrane layer 42) may be thinner than the portions that do include first porous layer 46 and second porous layer 48, respectively.

FIG. 4B illustrates an alternative configuration of antimicrobial accessory 26 about ICD 16, in which membrane layer 42 is positioned between first porous layer 46 and ICD 16 and between second porous layer 48 and ICD 16. In some examples, such a configuration may provide an increased elution rate compared to the configuration illustrated in FIG. 4A, as membrane layer 42 will not reduce an elution rate of antimicrobial from first porous layer 46 or second porous layer 48. Additionally or alternatively, antimicrobial accessory 26 may include an adhesive layer formed on membrane layer 42. Because membrane layer 42 may be mechanically stronger than first porous layer 46 or second porous layer 48, attaching antimicrobial accessory 26 to ICD 16 via an adhesive layer formed on membrane layer 42 may be preferable to attaching antimicrobial accessory 26 to ICD 16 via an adhesive layer formed on first porous layer 46 or second porous layer 48.

FIGS. 5A and 5B illustrate another example of a configuration of an antimicrobial accessory 92. In FIGS. 5A and 5B, antimicrobial accessory 92 is configured as a sheath that may be sized to fit intimately over housing 40 of ICD 16. In this way, antimicrobial accessory 92 may form a friction fit around housing 40 of ICD 16, which may assist with maintaining relative positioning of ICD 16 and antimicrobial accessory 26 after implantation of ICD 16 and accessory 26 in a body of a patient 12.

As shown in FIG. 5B, which is a plan view of ICD 16 and antimicrobial accessory 26, antimicrobial accessory 26 includes a membrane layer 94, a first porous layer 96, and a second porous layer 98. Membrane layer 94 includes a first major surface having a first lateral portion 100, a second lateral portion 102, a third lateral portion 104, and a fourth lateral portion 106. Second lateral portion 102 is between first lateral portion 100 and third lateral portion 104, as is fourth lateral portion 106. First porous layer 96 is formed on first lateral portion 100 and second porous layer 98 is formed on third lateral portion 104. Second lateral portion 102 and fourth lateral portion 106 do not include porous layers formed thereon.

In the example shown in FIG. 5B, antimicrobial accessory is disposed about ICD 16 such that first porous layer 96 is adjacent to first major side 108 of ICD 16 and second porous layer 98 is adjacent to second major side 110 of ICD 16. Such an orientation may improve spatial distribution of antimicrobial within the implant pocket as the antimicrobial eluted from antimicrobial accessory 26. Second lateral portion 102 of membrane layer 42 is adjacent a third side 112 of ICD 16 and fourth lateral portion 106 is adjacent a fourth side 114 of ICD 16. As illustrated in FIG. 5B, a volume occupied by each of second lateral portion 102 of membrane layer 42 and fourth lateral portion 106 of membrane layer 42, respectively, is less than a volume occupied by either of first lateral portion 100 and first porous layer 96 or third lateral portion 104 and second porous layer 98, respectively. In this way, the configuration of antimicrobial accessory 26 may reduce a volume occupied within the implant pocket compared to an antimicrobial accessory including a porous layer throughout the antimicrobial accessory, while still providing advantageous spatial coverage of antimicrobial elution within the implant pocket.

In some examples, antimicrobial accessory 26 may include an adhesive layer formed on at least one of second lateral portion 102, fourth lateral portion 106, a surface of first porous layer 96, and/or a surface of second porous layer 98. As described above, the adhesive layer may include a PSA, such as a silicone, acrylic, polyisobutylene PSA a cyanoacrylate, a PLGA-based PSA, or the like. A PSA may assist with attaching antimicrobial accessory 26 to housing 40 of ICD 16, in addition to or as an alternative to a friction fit between accessory 26 and housing 40. In some examples, antimicrobial accessory 26 may not include an adhesive layer.

Similar to in FIG. 4A, membrane layer 94 may in some examples function as an elution modulating layer and affect an elution rate of antimicrobial from first porous layer 96 and/or second porous layer 98. Additionally or alternatively, membrane layer 94 may in some examples include an antimicrobial disposed therein, which may be the same antimicrobial that is disposed in first porous layer 96 or second porous layer 98, or may be a different antimicrobial.

Although FIG. 5B illustrates antimicrobial accessory 26 disposed about housing 40 with first porous layer 96 and second porous layer 98 between the housing 40 and membrane layer 94, other configurations are possible. For example, similar to FIG. 4B antimicrobial accessory 92 may be disposed about housing 40 such that membrane layer 94 is between first porous layer 96 and housing 40 and between second porous layer 98 and housing 40. Again, such a configuration may provide an increased elution rate compared to the configuration illustrated in FIG. 4A, as membrane layer 94 will not affect an elution rate of antimicrobial from first porous layer 96 or second porous layer 98. Additionally or alternatively, antimicrobial accessory 92 may include an adhesive layer formed on membrane layer 94. Because membrane layer 94 may have greater mechanical integrity than first porous layer 96 or second porous layer 98, attaching antimicrobial accessory 92 to ICD 16 via an adhesive layer formed on membrane layer 94 may be preferable to attaching antimicrobial accessory 92 to housing 40 via an adhesive layer formed on first porous layer 96 or second porous layer 98.

The configurations of antimicrobial accessory 26, 92 illustrated herein are merely exemplary and are not limiting. For example, an antimicrobial accessory 26, 92 may include more than two porous layers formed on a membrane layer. In such examples, the porous layers may be formed on the membrane layer in a pattern that results in a desired coverage of antimicrobial elution and a desired physical form factor. Other examples and configuration are also contemplated and are within the scope of the falling claims.

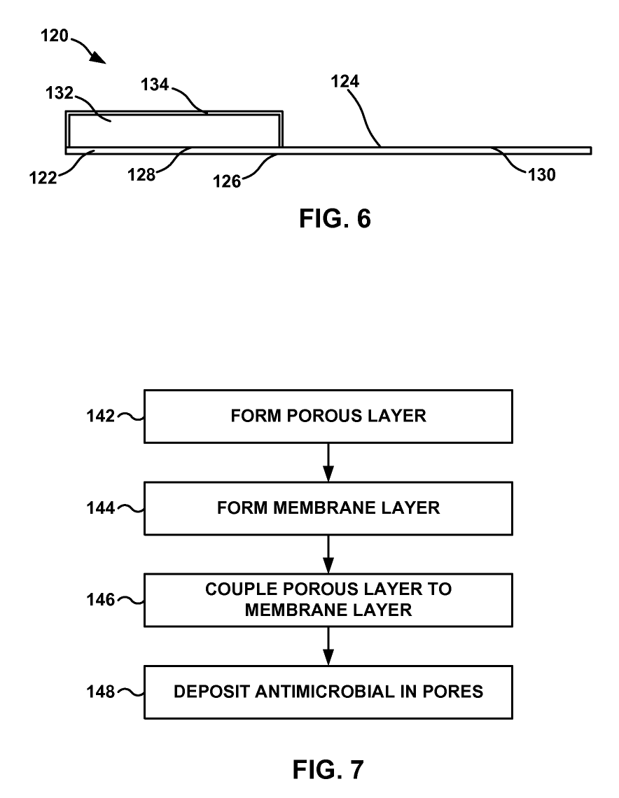

In some examples, an antimicrobial may not include first and second porous layers overlying different portions of a membrane layer. For example, as illustrated in FIG. 6, an antimicrobial accessory 120 may include a membrane layer 122 defining a first major surface 124 and a second major surface 126. First major surface 124 includes a first lateral portion 128 and a second lateral portion 130. Antimicrobial accessory 120 further includes a porous layer 132 overlying first lateral portion 128. Antimicrobial accessory 120 does not include a second porous layer, and no structure overlies second lateral portion 130 of antimicrobial accessory 120.

Porous layers 132 may include an antimicrobial disposed in pores of the porous layers 132. In some examples, porous layer 132 may include at least two antimicrobials.

In some examples, membrane layer 122 may also include an antimicrobial, which may be the same as or different than the antimicrobial disposed in the pores of porous layer 132. Membrane layer 122 may include substantially no antimicrobial, a lower concentration of antimicrobial than porous layer 132, or a greater concentration of antimicrobial than porous layer 132. By including an antimicrobial in membrane layer 122, greater control of an elution profile of the antimicrobial(s) in membrane layer 122 and porous layer 132 may be achieved compared to including antimicrobial(s) in only porous layer 132. For example, because membrane layer 122 is less porous than porous layer 132, the antimicrobial in membrane layer 122 may elute more slowly than antimicrobial in porous layer 132.

The antimicrobial in porous layer 132 and/or membrane layer 122 may include, for example, an antibiotic such as tetracyclines (e.g. minocycline, doxycycline), rifamycins (e.g. rifampin, rifaximin, rifapentine, rifabutin), macrolides (e.g. erythromycin), penicillins (e.g. nafcillin), cephalosporins (e.g. cefazolin), other beta-lactam antibiotics (e.g. imipenem, aztreonam) aminoglycosides (e.g. gentaminicn), glycopeptides (e.g. vancomycin, teicoplanin), quinolones (e.g. ciprofloxacin), fusidic acid, trimethoprim, metronidazole, mupirocin, polenes (e.g. amphotericin B), azoles (e.g. fluconazole) and beta-lactam inhibitors (e.g. sulbactam), tigecycline, daptomycin, clindamycin, or another fluoroquinolone, bacitracin, neomycin, an antiseptic, an antimicrobial peptide, a quaternary ammonium, or the like. In some examples, the antimicrobial may be provided in a salt form, e.g., gentamicin crobefate or gentamicin sulfate. In some examples, two or more antimicrobials may be selected to efficaciously prevent or treat any infection present proximate to the implant location of ICD 16, e.g., infection in the pocket in which ICD 16 is implanted. For example, gentamicin may be utilized alone or in combination with at least one other antimicrobial.

Membrane layer 122 and porous layer 132 may include a biodegradable or bioabsorbable polymer, such as, for example, at least one of collagen, PLGA, PLA, PGA, PEO, POE, PCL, poly(dioxanone), polyglyconate, hyaluronic acid, gelatin, fibrin, fibrinogen, cellulose, starch, cellulose acetate, PVP, a PEO/PPO copolymer, poly(ethylene vinyl acetate), poly(hydroxybutyrate-covalerate), polyanhydride, poly(glycolic acid-co-trimethylene carbonate), polyphosphoester, polyphosphoester urethane, a poly(amino acid), a cyanoacrylate, poly(trimethylene carbonate), poly(iminocarbonate), a copoly(ether-ester) such as PEO/PLA, a polyalkylene oxalate, a polyphasphazene, a polyarylate, a tyrosine0based biodegradable or bioabsorbably polymer, PHA, a sugar ester, or the like. In some examples, at least one of membrane layer 122 and porous layer 132 includes a mixture of at least two polymers. In some cases, membrane layer 122 may include a different polymer than porous layer 132. In other examples, membrane layer 122 and porous layer 132 may comprise the same polymer. The biodegradable or bioabsorbable polymer may degrade and be absorbed by the body of patient 12 over time after implantation of antimicrobial accessory 120 in the body of patient 12. This may be advantageous as it may ensure that substantially all of the antimicrobial is released from antimicrobial accessory 120, which may reduce risk of the growth or development of organisms that are resistant to the antimicrobial. Further, absorption of antimicrobial accessory 120 over time may remove a location for bacteria to grow.

In some examples, membrane layer 122 and porous layer 132 may comprise collagen, alone or in combination with at least one other polymer. The collagen may comprise collagen from an animal (xenogenous collagen) or from a human (autologous or allogenic collagen). The collagen may comprise at least one collagen type, such as Type-I, -II, -III, -IV, -VII, or -IX. Collagen Type-I may be obtained from animal tissue such as skin, tendons, or the like. In some examples, the collagen may be enzymatically treated prior to use, while in other examples, the collagen may not be enzymatically treated prior to use. Collagen may or may not be cross-linked.

Regardless of the particular polymer from which antimicrobial accessory 120 is formed, antimicrobial accessory 120 may include other components that may influence the properties of the accessory 120. For example, antimicrobial accessory 120 may include an antioxidant mixed in the polymer and/or the antimicrobial, which may reduce or substantially prevent oxidation of the antimicrobial. Exemplary antioxidants include, but are not limited to, monofunctional hindered phenolic antioxidants, such as those available under the trade designations Irganox 1076 and Irganox 1010 from Ciba Corp., Tarrytown, N.Y., butylated hydroxyl toluene (BHT), vitamin E, vitamin A, or vitamin C. In some examples, antimicrobial accessory 120 may include between approximately 0.1 wt. % and approximately 2 wt. % antioxidant.

Antimicrobial accessory 120 may optionally include an elution modulating layer 134 formed on porous layer 132. Elution modulating layer 134 may affect the rate at which antimicrobial is released from porous layer 132. In some examples, elution modulating layer 134 may comprise the same polymer as porous layer 132, while in other examples, elution modulating layer 134 includes a different polymer than porous layer 132. For example, the elution modulating layer may include a biodegradable polymer such as collagen, a polysaccharide, or the like. Elution modulating layer 134 may be applied to porous layer 132 by a variety of coating techniques, including, for example, spray coating, dip coating, extrusion, or the like.

Although not shown in FIG. 6, in some examples, second major surface 126 of membrane layer 122 or a surface of porous layer 132 may include formed thereon a layer of adhesive, such as a pressure sensitive adhesive (PSA). Suitable PSAs include, for example, silicone, acrylic, or polyisobutylene PSAs. In some examples, the PSA may be a bioresorbable polymer such as cyanoacrylate, a PLGA-based PSA, or the like. The adhesive layer may be applied to second major surface 126 or a surface of porous layer 132s by, for example, spray coating, knife coating, air knife coating, gap coating, gravure coating, slot die coating, metering rod coating, doctor blade, or the like. The adhesive layer may assist in attaching antimicrobial accessory 120 to an ICD 16. In other examples, antimicrobial accessory 120 may be self-adhesive (e.g., a polymer from which at least one of membrane layer 122 and/or porous layer 132 is formed may have adhesive properties when wet), antimicrobial accessory 120 may form a friction fit with surfaces of an ICD 16, or antimicrobial accessory 120 may be attached to an ICD 16 via a suture or other physical connection. In some examples, antimicrobial accessory 120 may not be attached to ICD 16 and may instead be implanted adjacent to ICD 16.

In some examples, second lateral portion 124 of membrane layer 122 may facilitate configuration and implantation of antimicrobial accessory 120 and ICD 16. For example, second lateral portion 124 may be wrapped about ICD 16 and/or at least one of leads 18, 20, 22. By wrapping of second lateral portion 124 about ICD 16 and/or at least one of leads 18, 20, 22, a relatively secure coupling between antimicrobial accessory 120 and ICD 16 a/or at least one of leads 18, 20, 22 may be effected.

FIG. 7 is a flow diagram illustrating an example of a method of forming an antimicrobial accessory, such as antimicrobial accessory 120 shown in FIG. 6. Initially, porous layer 132 may be formed as a porous sponge (142). In some examples, as described above, porous layer 132 comprises collagen. In such an example, porous layer 132 may be formed by first creating a dispersion or suspension comprising collagen in a solvent, such as water or another non-organic solvent. The dispersion or suspension may include between approximately 0.5 wt. % collagen and approximately 5 wt. % collagen and a balance solvent. The dispersion or suspension then may be freeze dried to form the collagen sponge.

Next, membrane layer 122 is formed (144). Membrane layer 122 may in some cases comprise collagen, as described above. In some examples in which membrane layer 122 comprises collagen, membrane layer 122 may be formed by applying mechanical pressure and heat to a collagen sponge wetted with an amount of solvent, such as water. For example, a collagen sponge may be wetted such that the solvent content is between approximately 2 wt. % and approximately 40 wt. % of the wetted sponge. The sponge may then be exposed to a temperature between approximately 50° C. and approximately 200° C. at a pressure between approximately 0.5 kg/cm² and approximately 1000 kg/cm² for between approximately 0.1 second and approximately 1 hour to reduce porosity of the sponge and form membrane layer 122.

In other examples in which membrane layer 122 comprises collagen, membrane layer 122 may be formed directly from a suspension or dispersion of collagen in a solvent. Again, the suspension or dispersion may comprise between approximately 0.5 wt. % and approximately 5 wt. % collagen and a balance solvent, such as water or another inorganic solvent, or an organic solvent. The suspension or dispersion may then be air dried to remove substantially all the solvent and form membrane layer 122. The porosity of the membrane layer 122 in such examples may be influenced by concentration of collagen in the suspension or dispersion, drying conditions, or the like.

Although not shown in FIG. 7, in some embodiments, when forming first porous layer 46 and/or second porous layer 48, the antimicrobial may be mixed in the solvent with the polymer. In this way, antimicrobial may be loaded in first porous layer 46 and/or second porous layer 48 in the same step in which the porous layer 46 and/or 48 is formed, instead of requiring a second step.

Porous layer 132 then may be coupled to membrane layer 122 (146). In some examples, porous layer 132 may be coupled to membrane layer 122 through use of pressure and elevated temperatures. Similar to the process described above for forming membrane layer 122, porous layer 132 and membrane layer 122 may be wetted with a solvent, such as water, to a moisture content of between approximately 2 wt. % and approximately 40 wt. % of the wetted sponge. Porous layer 132 then may be aligned with and disposed on first lateral portion 128 of membrane layer 122. Membrane layer 122 and porous layer 132 may then be exposed to heat and pressure, e.g., by use of a heated press. In some examples, porous layer 132 and membrane layer 122 may then be exposed to a temperature between approximately 50° C. and approximately 200° C. at a pressure between approximately 0.5 kg/cm² and approximately 1000 kg/cm² for between approximately 0.1 second and approximately 1 hour to reduce physically mechanically couple porous layer 132 to membrane layer 122.

Once porous layer 132 have been coupled to membrane layer 122, antimicrobial may be deposited in pores of porous layer 132 (148). For example, the antimicrobial may be dissolved in a solvent or mixture of solvents at an elevated temperature. The solvent may include, for example, water, methanol, ethanol, isopropyl alcohol, ethyl acetate, acetone, tetrahydrofuran, acetonitrile, heptane, methylene chloride, chloroform,or the like. Antimicrobial accessory 26 including porous layer 132 is then exposed to the solution of the antimicrobial in the solvent by, for example, submerging porous layer 132 in the antimicrobial solution or spraying the antimicrobial solution on porous layer 132. Once the pores of porous layer 132 are substantially filled with solution, the solution may be cooled, which initiates precipitation of the antimicrobial out of solution and deposition of the antimicrobial into the pores. Antimicrobial accessory 26 then may be dried in a vacuum to remove substantially all of the solvent.

In some examples, membrane layer 122 may also comprise an antimicrobial. For example, an antimicrobial may be deposited in membrane layer 122 during the same process during which the antimicrobial is deposited in at least one of porous layer 132. In other examples, membrane layer 122 may not include an antimicrobial, and membrane layer 122 may not be exposed to the antimicrobial solution when the antimicrobial is deposited in porous layer 132.

Although not illustrated in FIG. 7, in some examples, the method may optionally include at least one of coating porous layer 132 with an elution modulating layer 134, as described in step (70) of FIG. 3, coating second major surface 126 of membrane layer 122 with a layer of adhesive, as described in step (72) of FIG. 3, and/or sterilizing antimicrobial accessory 120, as described in step (74) of FIG. 3.

Various examples have been described in the disclosure. These and other examples are within the scope of the following claims.

1. An antimicrobial accessory comprising:
a membrane layer defining a major surface comprising a first lateral portion and a second lateral portion, wherein the membrane layer comprises a first biodegradable polymer; and
a porous layer overlying the first lateral portion, wherein the first porous layer comprises a second biodegradable polymer and an antimicrobial, and wherein the porous layer does not overlie the second lateral portion of the membrane layer.

2. The antimicrobial accessory of claim 1, wherein the membrane layer further comprises a third lateral portion, wherein the porous layer comprises a first porous layer, and wherein the antimicrobial accessory further comprises:
a second porous layer overlying the third lateral portion, wherein the second porous layer comprises a third biodegradable polymer and the antimicrobial, and wherein the second porous layer does not overlie the second lateral portion of the membrane layer.

3. The antimicrobial accessory of claim 1, wherein at least one of the first biodegradable polymer and the second biodegradable polymer, or the third biodegradable polymer comprises at least one of collagen, poly(lactic-co-glycolic acid), poly(lactic acid), poly(glycolic acid), poly(ethylene oxide), poly(ortho ester), poly(ε-caprolactone), poly(dioxanone), polyglyconate, hyaluronic acid, gelatin, fibrin, fibrinogen, cellulose, starch, cellulose acetate, polyvinylpyrrolidone, a poly(ethylene oxide)/poly(propylene oxide) copolymer, poly(ethylene vinyl acetate), poly(hydroxybutyrate-covalerate), polyanhydride, poly(glycolic acid-co-trimethylene carbonate), polyphosphoester, polyphosphoester urethane, a poly(amino acid), a cyanoacrylate, poly(trimethylene carbonate), poly(iminocarbonate), a copoly(ether-ester), a polyalkylene oxalate, a polyphasphazene, a polyarylate, a tyrosine-based polymer, polyhydroxyalkanoate, or a sugar ester.

4. The antimicrobial accessory of claim 1, wherein the first biodegradable polymer and the second biodegradable polymer comprise the same polymer.

5. The antimicrobial accessory of claim 4, wherein the first biodegradable polymer and the second biodegradable polymer each comprise collagen.

6. The antimicrobial accessory of claim 1, wherein the antimicrobial comprises at least one of minocycline, doxycycline, clindamycin, rifampin, rifaximin, rifapentine, rifabutin, tigecycline, daptomycin, gentamicin, vancomycin, teicoplanin or another fluoroquinolone, bacitracin, neomycin, an antiseptic, an antimicrobial peptide, or a quaternary ammonium.

7. The antimicrobial accessory of claim 6, wherein the antimicrobial comprises gentamicin.

8. The antimicrobial accessory of claim 1, wherein the antimicrobial comprises a first antimicrobial, and wherein the membrane layer further comprises a second antimicrobial.

9. The antimicrobial accessory of claim 8, wherein the second antimicrobial comprises gentamicin.

10. The antimicrobial accessory of claim 1, further comprising an elution modulating layer with or without antimicrobial overlying the porous layer.

11. The antimicrobial accessory of claim 1, wherein the major surface comprises a first major surface, wherein the membrane layer further comprises a second major surface opposite the first major surface, and wherein the antimicrobial accessory further comprises an adhesive layer overlying the second major surface.

12. The antimicrobial accessory of claim 1, wherein the membrane layer is substantially nonporous.

13. A kit comprising:
an implantable medical device; and
an antimicrobial accessory comprising:
a membrane layer defining a first major surface comprising a first lateral portion, a second lateral portion, and a third lateral portion, wherein the membrane layer comprises a first biodegradable polymer,
a first porous layer overlying the first lateral portion, wherein the first porous layer comprises a second biodegradable polymer and a first antimicrobial, and
a second porous layer overlying the second lateral portion, wherein the second porous layer comprises a third biodegradable polymer and a second antimicrobial.

14. The kit of claim 13, wherein at least one of the first biodegradable polymer, the second biodegradable polymer, or the third biodegradable polymer comprises at least one of collagen, poly(lactic-co-glycolic acid), poly(lactic acid), poly(glycolic acid), poly(ethylene oxide), poly(ortho ester), poly(ε-caprolactone), poly(dioxanone), polyglyconate, hyaluronic acid, gelatin, fibrin, fibrinogen, cellulose, starch, cellulose acetate, polyvinylpyrrolidone, a poly(ethylene oxide)/poly(propylene oxide) copolymer, poly(ethylene vinyl acetate), poly(hydroxybutyrate-covalerate), polyanhydride, poly(glycolic acid-co-trimethylene carbonate), polyphosphoester, polyphosphoester urethane, a poly(amino acid), a cyanoacrylate, poly(trimethylene carbonate), poly(iminocarbonate), a copoly(ether-ester), a polyalkylene oxalate, a polyphasphazene, a polyarylate, a tyrosine-based polymer, polyhydroxyalkanoate, or a sugar ester.

15. The kit of claim 13, wherein the first biodegradable polymer, the second biodegradable polymer, and the third biodegradable polymer comprise collagen.

16. The kit of claim 13, wherein at least one of the first antimicrobial and the second antimicrobial comprises at least one of minocycline, doxycycline, clindamycin, rifampin, rifaximin, rifapentine, rifabutin, tigecycline, daptomycin, gentamicin, vancomycin, teicoplanin or another fluoroquinolone, bacitracin, neomycin, an antiseptic, an antimicrobial peptide, or a quaternary ammonium.

17. The kit of claim 16, wherein the first antimicrobial and the second antimicrobial comprise gentamicin.

18. The kit of claim 13, wherein the membrane layer further comprises gentamicin.

19. The kit of claim 13, further comprising an elution modulating layer with or without antimicrobial overlying at least one of the first porous layer or the second porous layer.

20. The kit of claim 13, wherein the major surface comprises a first major surface, wherein the membrane layer further comprises a second major surface opposite the first major surface, and wherein the antimicrobial accessory further comprises an adhesive layer overlying the second major surface.

21. The kit of claim 13, wherein the membrane layer is substantially nonporous.

22. The kit of claim 13, wherein the antimicrobial accessory is configured to be coupled to the implantable medical device.

23. A method comprising:
forming a porous layer comprising a first biodegradable polymer;
forming a membrane layer comprising a second biodegradable polymer, wherein a major surface of the membrane layer comprises a first lateral portion and a second lateral portion;
coupling the porous layer to the first lateral portion, wherein the porous layer does not overlie the second lateral portion; and
depositing an antimicrobial in pores of the porous layer.

24. The method of claim 23, wherein forming the porous layer comprises:
creating a dispersion or suspension of collagen in a solvent; and
freeze drying the dispersion or suspension.

25. The method of claim 23, wherein forming the membrane layer comprises:
creating a dispersion or suspension of collagen in a solvent;
freeze drying the dispersion or suspension to form a collagen sponge; and
pressing the collagen sponge at an elevated temperature.

26. The method of claim 23, wherein forming the membrane layer comprises forming the membrane layer comprising collagen by:
creating a dispersion or suspension of collagen in a solvent;
air drying the dispersion or suspension.

27. The method of claim 23, wherein coupling the porous layer to the first lateral portion comprises:
aligning the porous layer with the first lateral portion;
disposing the porous layer on the first lateral portion; and
pressing the porous layer on the first lateral portion at an elevated temperature.

28. A method comprising:
forming a first porous layer comprising a first biodegradable polymer;
forming a second porous layer comprising a second biodegradable polymer;
forming a membrane layer comprising a third biodegradable polymer, wherein a major surface of the membrane layer comprises a first lateral portion, a second lateral portion, and a third lateral portion;
coupling the first porous layer to the first lateral portion;
coupling the second porous layer to the second lateral portion; and
depositing an antimicrobial in pores of the first porous layer and the second porous layer.

29. The method of claim 28, wherein forming the first porous layer comprises forming the first porous layer comprising collagen by:
creating a dispersion or suspension of collagen in a solvent; and
freeze drying the dispersion or suspension.

30. The method of claim 28, wherein forming the second porous layer comprises forming the second porous layer comprising collagen by:
creating a dispersion or suspension of collagen in a solvent; and
freeze drying the dispersion or suspension.

31. The method of claim 28, wherein forming the membrane layer comprises forming the membrane layer comprising collagen by:
creating a dispersion or suspension of collagen in a solvent;
freeze drying the dispersion or suspension to form a collagen sponge; and
pressing the collagen sponge at an elevated temperature.

32. The method of claim 28, wherein forming the membrane layer comprises forming the membrane layer comprising collagen by:
creating a dispersion or suspension of collagen in a solvent;
air drying the dispersion or suspension.

33. The method of claim 28, wherein coupling the first porous layer to the first lateral portion comprises:
aligning the first porous layer with the first lateral portion;
disposing the first porous layer on the first lateral portion; and
pressing the first porous layer on the first lateral portion at an elevated temperature.

34. The method of claim 28, wherein coupling the second porous layer to the second lateral portion comprises:
aligning the second porous layer with the second lateral portion;
disposing the second porous layer on the second lateral portion; and
pressing the second porous layer on the second lateral portion at an elevated temperature.

35. The method of claim 28, further comprising forming an elution modulating layer with or without antimicrobial on a surface of at least one of the first porous layer and the second porous layer.

36. The method of claim 28, wherein the major surface of the membrane layer comprises a first major surface, the method further comprising forming an adhesive layer on a second major surface of the membrane layer.

37. A method comprising:
forming a porous layer comprising a first biodegradable polymer and an antimicrobial;
forming a membrane layer comprising a second biodegradable polymer, wherein a major surface of the membrane layer comprises a first lateral portion and a second lateral portion; and
coupling the porous layer to the first lateral portion, wherein the porous layer does not overlie the second lateral portion.

38. The method of claim 37, wherein the first biodegradable polymer comprises collagen, and wherein forming the porous layer comprises:
creating a dispersion or suspension of collagen and the antimicrobial in a solvent; and
freeze drying the dispersion or suspension.

39. The method of claim 37, wherein the first biodegradable polymer comprises collagen, and wherein forming the membrane layer comprises:
creating a dispersion or suspension of collagen and the antimicrobial in a solvent;
freeze drying the dispersion or suspension to form a collagen sponge; and
pressing the collagen sponge at an elevated temperature.

40. The method of claim 37, wherein coupling the porous layer to the first lateral portion comprises:
aligning the porous layer with the first lateral portion;
disposing the porous layer on the first lateral portion; and
pressing the porous layer on the first lateral portion at an elevated temperature.
